(19) United States
(12) Patent Application Publication
(10) Pub. No.: US 2023/0364004 A1
(43) Pub. Date: Nov. 16, 2023

(54) STABLE COMPOSITIONS CONTAINING A CANNABINOID

(71) Applicant: ANGROW COMPANY LIMITED, North Kingstown, RI (US)

(72) Inventors: Dengfeng YANG, Shenzhen (CN); Xuan TAN, Birmingham, AL (US); Chunfeng CHAI, Shenzhen (CN)

(73) Assignee: ANGROW COMPANY LIMITED, North Kingstown, RI (US)

(21) Appl. No.: 18/317,047

(22) Filed: May 12, 2023

Related U.S. Application Data
(60) Provisional application No. 63/364,622, filed on May 12, 2022.

(51) Int. Cl. A61K 8/9789; A61K 8/34; A61K 8/37; A61Q 19/00

(52) U.S. Cl. CPC A61K 8/9789; A61K 8/347; A61K 8/342; A61K 8/345; A61K 8/37; A61Q 19/00; A61K 2800/522

(57) ABSTRACT

The present disclosure provides a stable cosmetic composition. The composition may include one or more cannabinoids at 0.05%-10% in total by weight of the composition. The composition may also include an emulsifier at 0.02%-20% by weight of the composition. The composition may further include a stabilizing agent which includes an antioxidant.

### CROSS-REFERENCE TO RELATED APPLICATIONS

This application claims priority to U.S. Provisional Application No. 63/364,622, filed on May 12, 2022, the contents of which are hereby incorporated by reference.

### TECHNICAL FIELD

The present disclosure generally relates to stable compositions containing a cannabinoid, and more particularly, compositions containing a stable cannabinoid.

### BACKGROUND

Cannabinoids (e.g., cannabidiol (CBD), cannabigerol (CBG), etc.) are used in therapeutics, personal care (e.g., skincare, haircare, etc.), health products, or the like. Some of these uses result from the anti-inflammatory and/or antioxidant effects that cannabinoids possess. However, cannabinoids are often sensitive to oxidation and light, resulting in low stability and difficulties in preservation, storage, and transportation, further limiting the application of cannabinoids, especially in personal care. Therefore, it is desirable to provide formulations that can stabilize cannabinoids and stable compositions containing cannabinoids.

### SUMMARY

According to some embodiments of the present disclosure, a composition having improved stability of a cannabinoid is provided. The composition may include a cannabinoid, a lipid, water, an emulsifier, or the like. The components of the composition may be mixed to form an oil-in-water composition by using a Nano lipid carrier or an oil-in-water emulsion.

In an aspect of the present disclosure, a stable cosmetic composition is provided. The composition may include one or more cannabinoids at 0.05%-10% in total by weight of the composition. The composition may also include an emulsifier at 0.02%-20% by weight of the composition. The composition may a stabilizing agent which includes an antioxidant.

In some embodiments, the one or more cannabinoids may include cannabidiol (CBD), cannabigerol (CBG), or a derivative thereof.

In some embodiments, the one or more cannabinoids may be at 0.5%-5% in total by weight of the composition.

In some embodiments, the one or more cannabinoids are at 1%-3% in total by weight of the composition.

In some embodiments, the one or more cannabinoids may include CBD at about 1% by weight of the composition and CBG at about 1% by weight of the composition.

In some embodiments, the antioxidant is pentaerythrityl tetra-di-t-butyl hydroxyhydrocinnamate (PTDTBH), butylated hydroxytoluene (BHT), or propyl gallate.

In some embodiments, the antioxidant may be at 0.01%-2% by weight of the composition.

In some embodiments, the antioxidant may be at 0.05%-1% by weight of the composition.

In some embodiments, the antioxidant may be PTDTBH at about 0.1% by weight of the composition.

In some embodiments, the emulsifier may be at 1%-5% by weight of the composition.

In some embodiments, the emulsifier may include at least one of cetyl alcohol, cetearyl alcohol, polypropylene glycol (PPG) polyether, or polyglycerol fatty acid ester.

In some embodiments, the composition may further include a vitamin, a liquid lipid, a solid lipid, a moisturizing agent, a thickener, a fragrance, a coloring agent, or an absorbent.

In some embodiments, the vitamin may be selected from the group consisting of vitamin A and vitamin E. The liquid lipid may be selected from the group consisting of triglyceride, isosorbide derivatives, and long chain straight alkane. The solid lipid may be selected from the group consisting of fatty alcohol ester, vegetable wax, and beeswax. The moisturizing agent may be selected from the group consisting of glycerin and squalane. The thickener may be selected from the group consisting of polyacrylate crosspolymer-6, proteins, alcohols, and silicones. The fragrance may be selected from the group consisting of perfume, cologne, and aftershave. The coloring agent may be selected from the group consisting of fluorescein, quinoline, triphenylmethane, titanium dioxide, iron oxide, chromium oxide, organic colorants, and particles of silver and gold. The absorbent may be selected from the group consisting of sodium polyacrylate, kaolin, talc powder, and magnesium oxide.

In some embodiments, the composition may further include a vitamin, a liquid lipid, a solid lipid, a moisturizing agent, a thickener, a fragrance, a coloring agent, and an absorbent.

In another aspect of the present disclosure, a stable cosmetic composition may include CBD at 0.5%-1.5% by weight of the composition; CBG at 0.5%-1.5% by weight of the composition; an emulsifier at 0.1%-5% by weight of the composition; and a stabilizing agent comprising PTDTBH at 0.05%-0.5% by weight of the composition, or BHT at 0.5%-2% by weight of the composition.

In some embodiments, the CBD may be at about 1% by weight of the composition, and the CBG may be at about 1% by weight of the composition.

In some embodiments, the stabilizing agent may include PTDTBH at about 0.1% by weight of the composition.

In some embodiments, the emulsifier includes cetyl alcohol at 0.5%-1.5% by weight of the composition. The composition may further include water at about 75%-85% by weight of the composition; glycerin at about 2.5%-3.5% by weight of the composition; dicaprylyl carbonate at about 2.5%-3.5% by weight of the composition.

In another aspect of the present disclosure, a stable cosmetic composition is provided. The composition may include water at about 80% by weight of the composition; CBD at about 1% by weight of the composition; CBG at about 1% by weight of the composition; PTDTBH at about 0.1% by weight of the composition; glycerin at about 3% by weight of the composition; cetyl alcohol at about 1% by weight of the composition; and dicaprylyl carbonate at about 3% by weight of the composition.

In some embodiments, the composition may further include a vitamin, a liquid lipid, a solid lipid, a moisturizing agent, a thickener, a fragrance, a coloring agent, or an absorbent.

### BRIEF DESCRIPTION OF THE DRAWINGS

The present disclosure is further described in terms of exemplary embodiments. These exemplary embodiments are described in detail with reference to the drawings. It should be noted that the drawings are not to scale. These embodiments are nonlimiting exemplary embodiments, in which like reference numerals represent similar structures throughout the several views of the drawings, and wherein:

FIG. 1 is a schematic diagram illustrating images of a processed sample S1′ and a positive control sample S8 according to some embodiments of the present disclosure;

FIG. 2 is a schematic diagram illustrating images of a processed sample S2′ and the positive control sample S8 according to some embodiments of the present disclosure;

FIG. 3 is a schematic diagram illustrating images of a processed sample S3′ and the positive control sample S8 according to some embodiments of the present disclosure;

FIG. 4 is a schematic diagram illustrating images of a processed sample S4′ and the positive control sample S8 according to some embodiments of the present disclosure;

FIG. 5 is a schematic diagram illustrating images of a processed sample S5′ and the positive control sample S8 according to some embodiments of the present disclosure;

FIG. 6 is a schematic diagram illustrating images of a processed sample S6′ and the positive control sample S8 according to some embodiments of the present disclosure; and

FIG. 7 is a schematic diagram illustrating images of a processed sample S7′ and the positive control sample S8 according to some embodiments of the present disclosure.

### DETAILED DESCRIPTION

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown but is to be accorded the widest scope consistent with the claims.

### Definitions

As used herein, the term “percentage (i.e., %)” may be by weight of the total composition, unless specifically stated otherwise. The term “ratio” may refer to a weight ratio, unless specifically stated otherwise. The number of significant digits conveys neither a limitation on the indicated amounts nor the accuracy of the measurements. All numerical amounts should be understood to be modified by the word “about” unless otherwise specifically indicated. As used herein, the term “about” and its grammatical equivalents in relation to a reference numerical value and its grammatical equivalents as used herein can include a range of values plus or minus 10% from that value, such as a range of values plus or minus 10%, 9%, 8%, 7%, 6%, 5%, 4%, 3%, 2% or 1% from that value. For example, in some embodiments, the amount of “about 10” includes amounts from 9 to 11; in some embodiments, the amount of “about 10” includes amounts from 9.5 to 10.5. All measurements should be understood to be made at 25° C. and at ambient conditions, where “ambient conditions” may refer to conditions under about one atmosphere of pressure and at about 50% relative humidity. All such weights as they pertain to listed ingredients are based on the active level and do not include carriers or by-products that may be included in commercially available materials, unless otherwise specified. All numeric ranges are inclusive of narrower ranges and combinable; delineated upper and lower range limits are interchangeable to create further ranges not explicitly delineated.

The terms “actives” and “active component(s)” may refer to compound(s) that, when applied to a target object, provide a benefit or improvement to the target object. The target object herein may include a user or a portion thereof (e.g., keratinous tissue of the user, a target region of the keratinous tissue, tooth, or the like, or any combination thereof. As used herein, the term “keratinous tissue” may refer to keratin-containing layers disposed as the outermost protective covering of mammals which includes, but is not limited to, skin, hair, nails, cuticles, etc. Accordingly, the actives and/or active component(s) herein may be used for skincare, haircare, fingernail care, oral care, the like, or any combination thereof.

The term “apply” or “application,” as used in reference to a composition, may refer to applying or spreading the composition onto a skin surface (such as the epidemics), the hair, a surface of a fingernail, the surface of the tooth, etc. of the user.

The term “derivative” may refer to a molecule similar to that of another one, but differing from it with respect to a certain functional moiety (e.g., esters, ethers, amides, amines, carboxylic acids, hydroxyls, acetyls, thiols, halogens, thiols, and/or salt derivatives of the relevant molecule).

The term “dermatologically acceptable” may refer to that the compositions or components thereof are suitable for use in contact with mammalian keratinous tissue without undue toxicity, incompatibility, instability, allergic response, and the like.

The term “effective amount” may refer to an amount of a compound or composition sufficient to significantly induce a positive benefit to keratinous tissue, such as health, appearance, and/or feel benefit, including, independently or in combination, the benefits disclosed herein, but low enough to avoid serious side effects (i.e., to provide a reasonable benefit to risk ratio, within the scope of sound judgment of the skilled artisan). An effective amount of a cannabinoid may be an amount sufficient to regulate a desired condition of mammalian keratinous tissue when topically applied thereto in a composition (e.g., a personal care composition) over the course of a treatment period.

The “personal care composition” may refer to a topical composition for regulating a condition of mammalian keratinous tissue (e.g., skin, hair, fingernails) and/or tooth. Exemplary personal care compositions may include skin creams, lotions, and serums; shave prep compositions; body washes; deodorants and antiperspirants, shampoos; conditioners; toothpaste, mouthwashes; or the like, or any combination thereof. As used herein, the term “regulating the condition of mammalian keratinous tissue and/or tooth” may refer to improving the appearance and/or feel of keratinous tissue and/or tooth.

The term “topical” may refer to a composition that is intended to be applied to a bodily surface such as skin or hair.

The term “cannabinoid containing compositions (e.g., personal care products)” may refer to any compositions that contain a cannabinoid. Preferred compositions may include compositions used for regulating the condition of the skin, even more preferably reducing the appearance of skin aging, reducing the appearance or occurrence of skin acne, and/or treating skin acne. The cannabinoid containing compositions herein may also exhibit an absence of significant (e.g., consumer-unacceptable) skin irritation and good aesthetics.

The term “signs of skin aging” may include, but is not limited to, all outward visibly and tactilely perceptible manifestations as well as any other macro or micro effects due to skin aging. Such signs may be induced or caused by intrinsic factors or extrinsic factors, e.g., chronological aging and/or environmental damage. These signs may result from processes that include, but are not limited to, the development of textural discontinuities such as wrinkles and coarse deep wrinkles, skin lines, crevices, bumps, large pores (e.g., associated with adnexal structures such as sweat gland ducts, sebaceous glands, or hair follicles), or unevenness or roughness, loss of skin elasticity (loss and/or inactivation of functional skin elastin), sagging (including puffiness in the eye area and jowls), loss of skin firmness, loss of skin tightness, loss of skin recoil from deformation, discoloration (including undereye circles), blotching, sallowness, hyperpigmented skin regions such as age spots and freckles, keratoses, abnormal differentiation, hyper keratinization, elastosis, collagen breakdown, and other histological changes in the stratum corneum, dermis, epidermis, the skin vascular system (e.g., telangiectasia or spider vessels), and underlying tissues, especially those proximate to the skin.

The term “stable,” when referring to a cannabinoid contained in a composition (e.g., a personal care composition), may refer to less than 45% (e.g., less than 25%, 20%, 15%, 10%, or even less than 5%) of the cannabinoid present in the composition degrades (i.e., chemically converted to a different compound via, for example, oxidation or some other chemical process) when the composition is subjected to environmental conditions commonly experienced or conditions that are used for assessing stability by compositions of the type (e.g., ambient temperature, 30° C., 35° C., or 40° C. for 2 or more weeks, 1 month or more, 2 months or more, or 3 months or more). In some embodiments, the cannabinoid contained in the composition may be stable at 45° C. for more than 2, 4, or even 8 weeks. A suitable method of determining cannabinoid stability may include a high performance liquid chromatography (HPLC) method.

Before the present discovery, stabilizing a cannabinoid (e.g., CBD, CBG, etc.) in compositions (e.g. solution or cosmetic composition) at room temperature was problematic. Taking CBD as an example, the stability of CBD may be influenced by multiple factors (e.g., temperature, oxidation, light, solvent, etc.). For example, CBD may be highly unstable at room temperature, while CBD may be relatively stable at 5° C. or lower temperature. As another example, CBD may be highly unstable in oxidizing and/or light environments, while CBD may be relatively stable when CBD is protected from oxidization and light. As still another example, CBD may be more stable in the solvent of non-aqueous medium (e.g., ethanol) than in the solvent of aqueous medium. As still another example, 10% of CBD may be degraded within 24 hours in a simulated physiological condition (PH 7.4 and 37° C.). The above descriptions may indicate that CBD is highly unstable, which needs to be taken into count in the development of compositions and products containing CBD.

The present disclosure provides compositions containing a cannabinoid exhibiting good stability. In order to measure the stability of a product, a certain stability criteria may be established. The certain stability criteria may be based on a percentage of cannabinoids (by mass) remaining in a composition or product after a given time at a given temperature. For example, if a cannabinoid is added to a product at 0.1%, and 0.0879% of the cannabinoid remained after three weeks of storage at 45° C., then such a product may be said to have retained 87% of the original cannabinoid after three weeks storage at 45° C.

Stability tests, such as shelf-life tests, may also be used to evaluate the stability of a composition (e.g., a personal care composition). For example, in order to evaluate the stability of a cannabinoid in a given composition, the composition may be placed in a container that limits the free flow of oxygen (e.g., an aluminum tube, laminated tube, glass jar, HDPE jar, PP jar, HDPE pump) and stored fora given time (e.g., 12 weeks) at a given temperature (e.g., constant 45° C.). The percentage loss of a cannabinoid may be measured after an elapsed time period.

Compositions

In the present disclosure, a composition having improved the stability of a cannabinoid is provided. The composition may include a cannabinoid, a lipid, water, an emulsifier, or the like. The composition may be an oil-in-water composition/formulation, such that the cannabinoid contained in the composition may be stable in environmental conditions and/or applied on mammalian keratinous tissue (e.g., skin, hair, fingernails) and/or tooth of the user. The cannabinoid may include CBD, CBG, or the like, or any combination thereof. For example, the cannabinoid may include only CBD or CBG. As another example, the cannabinoid may include a mixture of CBD and CBG.

In some embodiments, the composition (also referred to as a first composition) may be in the form of a stable Nano lipid carrier. The stable Nano lipid carrier may refer to that the cannabinoid remained in the first composition after three months of storage at 45° C. and in light may be greater than 55% by weight of the original cannabinoid in the first composition. In some embodiments, the cannabinoid may be at 0.05%-0.5% by weight of the first composition. For example, the cannabinoid of the first composition by weight may be at 0.05%, 0.1%, 0.2%, 0.3%, 0.4%, 0.5%, etc. In some embodiments, the lipid may be at 0.026%-15% by weight of the first composition. For example, the lipid of the first composition may include a liquid lipid and a solid lipid. The liquid lipid may include triglyceride, carbonate, long chain straight alkane, isosorbide derivatives (e.g., isosorbitan or isosorbide ether), or the like, or any combination thereof. The solid lipid may include fatty alcohol ester, vegetable wax, or the like, or any combination thereof. The liquid lipid of the first composition by weight may be at 0.01%-5%. The solid lipid of the first composition by weight may be at 0.016%-10%. In some embodiments, the emulsifier may be at 0.02%-20% by weight of the first composition. For example, the emulsifier of the first composition by weight may be at 0.02%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, etc. The emulsifier may include polypropylene glycol (PPG) polyether, polyglycerol fatty acid ester, or the like, or any combination thereof. In some embodiments, the weight ratio of the emulsifier to the lipid in the first composition may be (0.4-3.5):1. For example, the weight ratio of the emulsifier to the lipid in the first composition may be 0.4:1, 0.5:1, 0.9:1, 1:1, 1.5:1, 2:1, 2.5:1. 3:1, 3.5:1, etc. In some embodiments, the first composition may further include optional ingredients as described elsewhere in the present disclosure, e.g., including a thickening agent, a phenol, a perfume, a coloring agent, an active component, or the like, or any combination thereof. For example, the first composition may consist of the cannabinoid, the liquid lipid, the solid lipid, the emulsifier, the water, and the thickening agent. As another example, the first composition may consist of the cannabinoid, the liquid lipid, the solid lipid, the emulsifier, the water, and one or more active components.

In some embodiments, the first composition may be prepared according to the following operations. A first liquor may be obtained by melting, at a preset temperature, the solid lipid, the liquid lipid, and the emulsifier. The preset temperature may be 50° C. to 95° C. For example, the preset temperature may be 50° C., 55° C., 60° C. 65° C., 70° C., 80° C., 80° C., 85° C., 90° C., 95° C., etc. A second liquor may be obtained by mixing the cannabinoid and the first liquor. The second liquor may be in a water-in-oil form. A third liquor may be obtained by mixing the water at the preset temperature and the second liquor. The third liquor may be in an oil-in-water form. A Nano lipid carrier may be obtained by cooling the third liquor to room temperature. It should be noted that conditions (e.g., the temperature or order of the operations) of the preparation process may be adjusted according to different situations, which is not limiting.

In some embodiments, the composition (also referred to as a second composition) may be in the form of a stable oil-in-water (O/W) emulsion. The stable O/W emulsion may include a continuous aqueous phase, which typically includes water, water-miscible liquids, and/or water-soluble materials, and a dispersed hydrophobic phase, which typically includes lipids, oils, and/or oily materials. The O/W emulsions herein may include 1% to 50% (e.g., 1% to 30%) by volume of the dispersed hydrophobic phase and 1% to 98% (e.g., 40% to 90%) by volume of the continuous hydrophilic phase. The emulsion may also include a gel network, such as described in G. M. Eccleston, Application of Emulsion Stability Theories to Mobile and Semisolid OAV Emulsions, Cosmetics & Toiletries, Vol. 101, November 1996, pp. 73-92.

The second composition may include a dermatologically acceptable carrier that enables other components (e.g., actives) to be delivered to the skin at an appropriate concentration. The dermatologically acceptable carrier may thus act as a diluent, dispersant, solvent, or the like for particulate material, which helps ensure that it can be applied to and distributed evenly over the selected target at an appropriate concentration. The dermatologically acceptable carrier may contain one or more dermatologically acceptable solid, semi-solid or liquid fillers, diluents, solvents, extenders, and the like. The dermatologically acceptable carrier may be solid, semi-solid, or liquid. In some instances, the carrier can be inert or it can provide benefits of its own to keratinous tissue. Concentrations of the dermatologically acceptable carrier can vary with the carrier selected and the intended concentrations of the composition components.

The type of the dermatologically acceptable carrier utilized in the present personal care composition depends on the type of product form desired for the composition. The topical composition useful in the present disclosure may be made into a wide variety of product forms such as are known in the art. The product forms may include, but are not limited to, lotions, creams, gels, sticks, sprays, ointments, pastes, mousses and cosmetics (e.g., solid, semi-solid, or liquid makeup, including foundations, eye-makeup, pigmented or non-pigmented lip treatments, e.g., lipsticks, and the like). The product forms may comprise several types of dermatologically acceptable carriers including, but not limited to, solutions, aerosols, emulsions, gels, solids, and liposomes.

The dermatologically acceptable carriers herein may contain a dermatologically acceptable, hydrophilic diluent. As used herein, “diluent” may include materials in which the particulate material of the composition can be dispersed, dissolved, or otherwise incorporated. Nonlimiting examples of hydrophilic diluents are water, organic hydrophilic diluents such as lower monovalent alcohols (e.g., C₁-C₄) and low molecular weight glycols and polyols, including propylene glycol, polyethylene glycol (e.g., Molecular weight 200-600 g/mole), polypropylene glycol (e.g., Molecular Weight 425-2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers, or the like, or any combination thereof. Water is a particularly suitable diluent. For example, the second composition may include about 60% to about 99.99% of the hydrophilic diluent.

The second composition may be made by conventional methods of making personal care compositions comprising an O/W emulsion system, which are known to those skilled in the art. In some embodiments, the second composition may include the cannabinoid, the lipid, the water, the emulsifier, an oil, and a fatty alcohol. The lipid (and/or the oil) and the water in the second composition may help to form an O/W emulsion. In some embodiments, the cannabinoid in the second composition may be stable. For example, less than 25% (e.g., less than 20%, 15%, less than 10%, etc.) of the cannabinoid in the second composition may be lost (i.e., degraded) between the time the second composition is packaged by the manufacturer and the time the package is first opened by a consumer. A suitable method for determining the amount of cannabinoid loss may include an HPLC method.

In some embodiments, the cannabinoid may be at 0.0001% to 2% (e.g., 0.005% to 2%, 0.01% to 1%, 0.01% to 0.5%) by weight of the second composition. For example, the cannabinoid of the second composition by weight may be at 0.05% to 0.5%. The cannabinoid of the second composition may be one cannabinoid (e.g., CBD or CBG) or a mixture of more than one cannabinoid (e.g., a mixture of CBD and CBG).

In some embodiments, the oil may be at 1% to 50% by weight of the second composition. The oil in the second composition may be with a hydrophile-lipophile balance (HLB) value of between 6 and 11.5. The HLB value of the oil may help to ensure that the cannabinoid and other oil-soluble actives are fully soluble in the oil and to help maintain proper portioning of the oil droplets in the O/W emulsion. In some embodiments, the oil may be an emollient (i.e., a water-immiscible, oily or waxy material used to increase the occlusive properties of skin), but need not necessarily be. It is believed, without being limited by theory, that certain oils can help stabilize the cannabinoid in an O/W emulsion by increasing cannabinoid partitioning in the oil droplets of the internal phase, thereby reducing the interaction between the cannabinoid and water present in the continuous phase of the emulsion. Thus, it may be important to ensure that a suitable amount of oil is present in the second composition and the ratio of oil relative to other ingredients is within a particular range. For example, the weight ratio of the oil to the cannabinoid in the second composition may be (5-50):1 (e.g., (15-35):1, (18-30):1).

In some embodiments, the oil in the second composition may include capric/caprylic triglyceride, coconut oil, argan oil, avocado oil, jojoba oil, moringa oil, soybean oil, Butyrospermum parkii (shea) nut extract, Vitis vinifera (grape) seed oil, Crambe abyssinica seed oil, Raphanus sativus (radish) seed extract, Argania spinose kernel oil extract, Limnanthes alba (meadowfoam) seed oil, Triticum vulgare (wheat) germ oil, Olea europaea (olive) fruit oil, hydrogenate vegetable oils, shea butter ethyl esters, canola oil, c10-18 triglyceride, hydrogenate palm oil, hydrogenate coco-glyceride, Cucurbita pepo (pumpkin) seed oil, Sunflower seed oil, rose hip oil, safflower oil, tea tree oil, lavender oil, flaxseed oil, isoamyl cocoate, phenoxyethyl caprylate, pentaerythrityl tetraethylhexanoate, pentaerythrityl tetraisostearate, glycereth-5 lactate, c12-15 alkyl benzoate, bis-phenylpropyl dimethicone, cyclopentasiloxane, octyldecanol, hexyldecanol, heptyl undecylenate, dimyristyl tartrate, hydrogenate polydecene, propylene glycol ricinoleate, peg-7 cocoate, decyltetradecanol, isopropyl lauroyl sarcosinate, octyldodecyl myristate, diisooctyl adipate, diisostearyl malate, hydrogenate castor oil, squalane, isohexadecane, ppg-15 stearyl ether, isopropyl myristate, phenyl trimethicone, propandiol diisostearate, dimethicone, dicaprylyl ether, octyl salicylate, isopropyl isostearate, or the like, or any combination thereof.

In some embodiments, the emulsifier contained in the second composition may help to stabilize the O/W emulsion. The emulsifier may be nonionic, anionic, or cationic. In some embodiments, the emulsifier may include cetearyl glucoside, cetearyl alcohol, or the like, or any combination thereof. In some embodiments, the emulsifier may be at 1% to 10% (e.g., 2% to 5%) by weight of the second composition. The amount and type of the emulsifier selected for use in the second composition may vary based on the type and amount of the oil in the second composition. For example, the second composition may have a ratio of the oil to the emulsifier of between (4-50):1 (e.g., (5-30):1, (10-20):1, (10-15):1, etc.).

In some embodiments, the fatty alcohol contained in the second composition may function as an emollient, emulsifier, thickener, structural agent, and/or carrying agent for oil soluble ingredients in the composition. The fatty alcohol may include cetyl alcohol, stearyl alcohol, cetearyl alcohol, behenyl alcohol, arachidyl alcohol, lignocaryl alcohol, or the like, or any combination thereof. It is believed, without being limited by theory, that the combination of emulsifier, fatty alcohol, and solvent creates a lamellar liquid crystal gel network in which the droplets of cannabinoid/oil are formed. Thus, it may be important to provide a suitable ratio of emulsifier to fatty alcohol in the second composition to produce a stronger lamellar gel-network/liquid crystal that makes less water available to react with the cannabinoid. For example, a weight ratio of the fatty alcohol to the emulsifier may be between 1:1 and 11:1 (e.g., between 5:3 and 10:1).

In some embodiments, the second composition may include a water-swellable material that acts to bind the water in the second composition. By binding some of the water in the second composition the interaction between the water and the cannabinoid may be reduced. The water-swellable material in the second composition may be natural or synthetic (e.g., water-swellable clay and superabsorbent polymers). The water-swellable material may be at 0.01% to 5% by weight of the second composition.

In some instances, the water-swellable material may be a superabsorbent polymer (“SAP”) present in the aqueous phase of the second composition as a multitude of particles. When swollen, the SAP may provide a light, cool, and silky feel during the application of the second composition. The SAP particles may have a dry, number-average particle size of 100 μm or less (e.g., 50 μm or less), for example, 2 μm to 100 μm, with a median particle size of 25, or even in the range of 2 μm to 40 μm with a median particle size of 12 μm. The SAP particles may have a water-absorbing capacity ranging from 20 to 2000 times their own weight (i.e., 20 g to 2000 g of water absorbed per gram of absorbent polymer), for example, 30 to 1500 times, 50 to 1000 times, or even 400 times. The water-absorbing characteristics of the SAP particles herein may be defined at standard temperature and pressure conditions for distilled water. For example, the value of the water-absorbing capacity of SAP herein may be determined by dispersing 0.5 g of polymer(s) in 150 g of distilled water, waiting 20 minutes, filtering the non-absorbed solution through a 150 μm filter for 20 minutes, and weighing the non-absorbed water to determine how much was absorbed by the polymer. %). In some instances, the viscosity of an SAP solution in 1% distilled water is in the range of 20 to 30 Pas (e.g., 22 to 29 Pas) at pH 4 and in the range of 23 to 28 Pas at pH 7. Once hydrated, the SAP particles in the second composition may swell to form relatively soft beads that have a number average diameter of 10 μm to 150 μm (e.g., 20 μm to 130 μm, 30 μm-120 μm, 40 μm-100 μm, 50 μm-90 μm, or even about 70 μm).

In some embodiments, the first or second composition may include a variety of optional ingredients (e.g., active components) that are known for use in personal care composition, as long as the optional ingredient(s) do not unduly alter product stability, aesthetics, or performance. The optional ingredients, when incorporated into the first or second composition, should be suitable for contact with human keratinous tissue without undue toxicity, incompatibility, instability, allergic response, and the like within the scope of sound judgment. The optional ingredients may be from about 0.0001% to about 50%; from about 0.001% to about 20%; from about 0.01% to about 10%; etc., by weight of the first or second composition. The optional ingredients may include abrasives, absorbents, opacifying agents, colorings/colorants (e.g., pigments, dyes, and lakes), particles, essential oils, anti-caking agents, foaming agents, anti-foaming agents, oil control agents, binders, biological additives, vitamins, minerals, peptides, sugar amines, flavonoid compounds, anti-oxidants, preservatives, phytosterols, protease inhibitors, tyrosinase inhibitors, exfoliating agents, skin lightening agents, sunless tanning agents, thickeners, pH adjusters, anti-acne actives, anti-cellulite actives, anti-wrinkle actives, phytosterols and/or plant hormones, N-acyl amino acid compounds, antimicrobials, antifungals, moisturizers, emollients, hum ectants, lubricating agents, fragrances, anti-dandruff agents, buffering agents, bulking agents, chelating agents, biocides, denaturants, astringents, external analgesics, anti-inflammatory agents, sunscreen agents, film formers and/or polymers for aiding film-forming properties and substantivity of the composition, propellants, reducing agents, sequestrants, conditioning agents, or the like, or any combination thereof. For example, the optional ingredient(s) of the second composition may include vitamins, proteins, zeolites, peptides, skin-lightening, sunscreen actives, terpene alcohols, desquamation actives, antiacne actives, anti-wrinkle actives, anti-atrophy actives, anti-oxidants, flavonoids, anti-inflammatory agents, anti-cellulite agents, tanning actives, skin-soothing actives, skin healing actives, conditioning agents, or the like, or any combination thereof.

In some embodiments, the first composition and/or the second composition may be useful for regulating the condition of skin and/or hair while maintaining good stability. Regulating a condition of the skin may include reducing the appearance of fine lines and/or wrinkles on the skin, reducing the appearance of eye bags and dark circles under the eyes, sagging skin, scars/marks, dimples, pores, stretch marks, roughness, skin surface blemishes, frown lines, expression lines, rhytides, blemishes, photodamage, crevices, and/or unevenness. Regulating the condition of the skin also includes reducing the occurrence and/or appearance of acne, treating acne, or the like.

The use of the first and/or second composition may include identifying a target portion of keratinous tissue (e.g., a facial skin surface such as the forehead, perioral, chin, periorbital, nose, and/or cheek) in need of treatment and/or where treatment is desired; and applying a safe and effective amount of the first and/or second composition to the target portion of tissue. Without intending to be bound by theory, it is believed that the application of an effective amount of the first and/or second composition to a target portion of keratinous tissue in need of treatment or where treatment is desired can provide the desired appearance benefit over the course of a treatment period.

The treatment period should be of sufficient time for the component(s) in the first and/or second composition to provide the desired benefit to the target portion of keratinous tissue (e.g., improve appearance, increase moisturization, etc.). The treatment period may last for at least 1 week (e.g., about 2 weeks, 4 weeks, 8 weeks, or even 12 weeks). For example, the treatment period may extend over multiple months (i.e., 3-12 months) or multiple years. As another example, the first and/or second composition may be applied most days of the week (e.g., at least 4, 5, or 6 days a week), at least once a day, or even twice a day during a treatment period of at least 2 weeks, 4 weeks, 8 weeks, or 12 weeks.

The step of applying the first and/or second composition may be accomplished by localized application. In reference to the application of a composition, the terms “localized,” “local,” or “locally” may refer to that the composition is delivered to the targeted area (e.g., a hyperpigmented portion of the skin) while minimizing delivery to keratinous surfaces where treatment is not desired. For example, the first and/or second composition may be applied and lightly massaged into an area of skin. The form of the first and/or second composition or the dermatologically acceptable carrier should be selected to facilitate localized application. While certain embodiments herein contemplate applying a composition locally to an area, it should be noted that compositions herein may be applied more generally or broadly to one or more skin surfaces.

The first and/or second composition may be applied in a variety of manners, including by rubbing, wiping, or dabbing with hands or fingers, or by an implement and/or delivery enhancement device. Exemplary implements may include a sponge or sponge-tipped applicator, a swab (for example, a cotton-tipped swab), a pen optionally comprising a foam or sponge applicator, a brush, a wipe, or the like, or any combination thereof. Exemplary delivery enhancement devices may include magnetic, mechanical, electrical, ultrasonic, and/or other energy devices. For example, the first and/or second composition may be spread onto the skin to facilitate the separation of the aqueous phase from the oil phase. When the aqueous and oil phases have separated, the first and/or second composition may be left on the keratinous tissue. Alternatively, the first and/or second composition may be allowed to remain on the skin for 5 seconds, 10 seconds, 30 seconds, or 1 minute before being rubbed into the keratinous tissue.

In some embodiments, the composition (also referred to as a third composition) may be in the form of a stable cosmetic composition. The third composition (i.e., the stable cosmetic composition) may include one or more cannabinoids, an emulsifier, a stabilizing agent which includes an antioxidant, etc. Under the action of the stabilizing agent, the cannabinoid(s) remained in the third composition after three months of storage at 45° C. and in light may be greater than 80% (e.g., 85%, 87%, 91%, etc.) by weight of the original cannabinoid(s) in the third composition.

In some embodiments, the one or more cannabinoids may be at 0.05%-10% in total by weight of the third composition. For example, the one or more cannabinoids of the third composition may be at 0.05%-5% (e.g., 0.05%, 0.1%, 0.2%, 0.3%, 0.4%, 0.5%, 1%, 2%, 3%, 4%, 5%, etc.) in total by weight of the third composition. As another example, the one or more cannabinoids of the third composition may be at 1%-3% (e.g., 1%, 1.5%, 2%, 2.5%, 3%, etc.) in total by weight of the third composition. In some embodiments, the one or more cannabinoids may include cannabidiol (CBD), cannabigerol (CBG), or a derivative thereof. For example, the third composition may include CBD and CBG which are at 2% in total by weight of the third composition. For instance, the third composition may include CBD at about 1% by weight of the third composition and CBG at about 1% by weight of the third composition.

In some embodiments, the emulsifier may be at 0.02%-20% by weight of the third composition. For example, the emulsifier may be at 1%-10% by weight of the third composition. As another example, the emulsifier may be at 1%-5% by weight of the third composition. For instance, the emulsifier may be at 0.02%, 0.1%, 0.5%, 1%, 1.5%, 2%, 3%, 4%, 5%, 6%, 7%, 8%, 9%, 10%, 11%, 12%, 13%, 14%, 15%, 16%, 17%, 18%, 19%, 20%, etc., by weight of the third composition. In some embodiments, the emulsifier may include cetyl alcohol, cetearyl alcohol, behenyl alcohol, stearic acid, stearyl alcohol, glycerin, polypropylene glycol (PPG) polyether, polyglycerol fatty acid ester, cetearyl olivate, sorbitan olivate, lecithin, PEG-7 Glyceryl Cocoate, or the like, or any combination thereof.

In some embodiments, the antioxidant may be at 0.01%-2% by weight of the third composition. For example, the antioxidant may be at 0.05%-1% by weight of the third composition. In some embodiments, the antioxidant may include pentaerythrityl tetra-di-t-butyl hydroxyhydrocinnamate (PTDTBH), butylated hydroxytoluene (BHT), propyl gallate, or the like, or any combination thereof. For example, the third composition may include PTDTBH at 0.01-0.2% by weight of the third composition. As another example, the third composition may include PTDTBH at about 0.2% by weight of the third composition. As still another example, the third composition may include BHT at about 1% by weight of the third composition. As further another example, the third composition may include propyl gallate at about 0.05-0.2% by weight of the third composition.

In some embodiments, the third composition may further include a vitamin, a liquid lipid, a solid lipid, a moisturizing agent, a thickener, a fragrance, a coloring agent, an absorbent, a preservative, or other active agents, or any combination thereof. The vitamin may be selected from the group consisting of vitamin A, vitamin B, vitamin C, vitamin E, vitamin H, vitamin K, etc. The liquid lipid may be selected from the group of triglyceride, carbonate, long chain straight alkane, isosorbide derivatives (e.g., isosorbitan or isosorbide ether), etc. The solid lipid may be selected from the group consisting of fatty alcohol ester, vegetable wax, beeswax, etc. The moisturizing agent (e.g., a humectant, an emollient, etc.) may be selected from the group consisting of glycerin, squalane, alpha-hydroxy acids (AHAs), hyaluronic acid, amino acid, collagen, ceramide, etc. The thickener may be selected from the group consisting of polyacrylate crosspolymer-6, polysaccharides, proteins, alcohols, silicones, waxes, etc. The fragrance may be selected from the group consisting of perfume, cologne, aftershave, etc. The coloring agent (e.g., a dye or a pigment) may be selected from the group consisting of fluorescein, quinoline, triphenylmethane, titanium dioxide, iron oxide, chromium oxide, organic colorants, particles of silver and gold, etc. The absorbent may be selected from the group consisting of sodium polyacrylate, kaolin, talc powder, magnesium oxide, etc. The preservative may be from the group consisting of phenoxyethanol, ethylhexyl glycerin, caprylhydroxamic acid, caprylyl glycol, etc. In some embodiments, the liquid lipid may be at 0.01%-5% by weight of the third composition. The solid lipid may be at 0.016%-10% by weight of the third composition. It should be noted that the third composition may also include any other active ingredients, which are not limited herein.

Merely by way of example, the third composition may include CBD at 0.5%-1.5% (e.g., 1%) by weight of the third composition, CBG at 0.5%-1.5% (e.g., 1%) by weight of the third composition, an emulsifier at 0.1%-5% by weight of the third composition; and a stabilizing agent comprising PTDTBH at 0.05%-0.5% (e.g., 0.1%) by weight of the third composition, or BHT at 0.5%-2% by weight of the third composition. The emulsifier at 1%-5% by weight of the third composition may include cetyl alcohol at 0.5%-1.5% by weight of the third composition, and the third composition may further include water at about 75%-85% by weight of the third composition, glycerin at about 2.5%-3.5% by weight of the third composition, dicaprylyl carbonate at about 2.5%-3.5% by weight of the third composition.

As another example, the third composition may include water at about 80% by weight of the third composition, CBD at about 1% by weight of the third composition, CBG at about 1% by weight of the third composition, PTDTBH at about 0.1% by weight of the third composition, glycerin at about 3% by weight of the third composition, cetyl alcohol at about 1% by weight of the third composition, and dicaprylyl carbonate at about 3% by weight of the third composition. The third composition may also include a vitamin, a liquid lipid, a solid lipid, a moisturizing agent, a thickener, a fragrance, a coloring agent, an absorbent, or the like, or any combination thereof.

In some embodiments, the usage/appliance of the third composition may be the same as or similar to the first composition and/or the second composition. For example, the third composition may be used as eye cream, body lotion, hand lotion, body cream, body butter, revitalizing lotion, or the like, or any combination thereof. For illustration purposes, some exemplary third compositions (i.e., examples 1-21) are listed herein with their ingredients and levels thereof by weight in corresponding third compositions.

### Example 1: Eye Cream

| | Level | |
|---|---|---|
| Ingredient | (%) | Function |
| De-ionized Water | 26.85 | Solvent |
| Glycerin | 5 | Moisturizing |
| | | agent |
| Propylene Glycol (And) Diazolidinyl Urea (And) | 0.3 | Preservative |
| Methylparaben (And) Propylparaben | | |
| Allantoin | 0.3 | Moisturizing |
| | | agent |
| Polysorbate 20 | 0.2 | Emulsifier |
| Water (And) Palmitoyl Hydroxypropyltrimonium | 0.15 | Emulsifier |
| Amylopectin/Glycerin Crosspolymer (And) Phenoxyethanol | | |
| (And) Tocopherol (And) Parabens (And) Hydrogenated | | |
| Lecithin | | |
| Cyclopentasiloxane | 37 | Thickener |
| Cyclopentasiloxane (And) Dimethicone/Vinyl Dimethicone | 19 | Preservative |
| Crosspolymer | | |
| Cetyl PEG/PPG-10/1 Dimethicone | 2 | Emulsifier |
| Silica | 1 | Active agent |
| Trifluoropropyl Dimethicone (And) Water (And) Glycerin | 3 | Active agent |
| (And) Pentylene Glycol (And) Phenoxyethanol (And) | | |
| Carbomer (And) Amodimethicone (And) Sodium Hydroxide | | |
| (And) Disodium EDTA | | |
| Dimethicone (And) Water (And) Glycerin (And) Pentylene | 3 | Active agent |
| Glycol (And) Dimethicone/Vinyl Dimethicone Crosspolymer | | |
| (And) Amodimethicone (And) Carbomer (And) | | |
| Phenoxyethanol (And) Sodium Hydroxide (And) | | |
| Disodium Edta | | |
| CBD | 1 | Cannabinoid |
| CBG | 1 | Cannabinoid |
| PTDTBH | 0.2 | Stabilizing |
| | | agent |

### Example 2: Body Location

| Ingredient | Level(%) |
|---|---|
| De-ionized Water | 82.85 |
| Glycerin | 2 |
| Disodium EDTA | 0.05 |
| Acrylates/Vinyl Isodecanoate Crosspolymer | 0.5 |
| Safflower Oil | 4 |
| Jojoba Oil | 3 |
| PEG-7 Glyceryl Cocoate | 1 |
| Cetyl Alcohol | 1.5 |
| Tocopheryl Acetate | 0.1 |
| Sodium Hydroxide | 1 |
| Porphyridium Polysaccharide | 1 |
| Caprylyl Glycol (and) Propylene Glycol (and) Glycerin | 0.8 |
| (and) Citrus Reticulata Fruit Extract (and) Citrus | |
| Aurantium (and) Amara Fruit Extract (and) Citrus Sinensis | |
| Peel Extract (and) Ascorbic Acid (and) Citric Acid | |
| (and) Lactic Acid (and) Water | |
| CBD | 1 |
| CBG | 1 |
| PTDTBH | 0.2 |

### Example 3: Hand Lotion

| Ingredient | Level(%) |
|---|---|
| De-ionized Water | 72.2 |
| Glycerin | 1 |
| 1,2 Hexanediol (and) Caprylyl Glycol | 2.5 |
| Disodium EDTA | 1 |
| Polyacrylate-13 (and) Polyisobutene (and) Polysorbate 20 | 2 |
| Petrolatum | 10 |
| Tocopheryl Acetate | 0.1 |
| Jojoba Oil | 1 |
| Ethylhexyl Ethylhexanoate | 8 |
| CBD | 1 |
| CBG | 1 |
| PTDTBH | 0.2 |

### Example 4: Body Cream

| Ingredient | Level(%) |
|---|---|
| De-ionized Water | 53.8 |
| Glycerin | 2 |
| Butylene Glycol | 8 |
| Methylisothiazolinone (and) Chlorphenesin | 1 |
| Water (and) Glycerine (and) Butylene Glycol (and) Zea Mays | 4 |
| (Corn) Starch (and) Natto Gum | |
| Hydrogenated Poly(C6-14 Olefin) (and) Olea Europa (Olive) | 3 |
| Fruit Extract (and) Beta-Sitosterol (and) Tocopherol | |
| Butyrospermum Parkii (Shea Butter) Extract (and) | 3 |
| Limnanthes Alba (Meadowfoam) Seed Oil | |
| Hydrogenated Polyisobutene | 5 |
| Hydrogenated Polyisobutene | 3 |
| Hydrogenated Polyisobutene (and) Dimethicone (and) | 8 |
| Polyethylacrylate | |
| Polymethyl Methacrylate | 3 |
| Polyacrylamide (and) Hydrogenated Polydecene (and) | 4 |
| Ethylene/Propylene Copolymer (and) Polyglyceryl- 10 | |
| Stearate | |
| CBD | 1 |
| CBG | 1 |
| PTDTBH | 0.2 |

### Example 5: Facial Lotion

| Ingredient | Level(%) |
|---|---|
| De-ionized Water | 66.6 |
| Glycerin | 5 |
| Acrylates/C10-30 Alkyl Acrylate Crosspolymer | 0.2 |
| Phenoxyethanol (and) Ethylhexylglycerin | 0.5 |
| Isopropyl Isostearate | 7 |
| Caprylic/Capric Triglyceride | 7 |
| Cyclopentasiloxane | 5 |
| Prunus Amygdalus Dulcis (Sweet Almond) Oil | 4 |
| Behenyl Alcohol | 2 |
| Methyl Glucose Sesquistearate | 0.4 |
| Tocopheryl Acetate | 0.1 |
| CBD | 1 |
| CBG | 1 |
| PTDTBH | 0.2 |

### Example 6: Body Cream

| Ingredient | Level(%) |
|---|---|
| De-ionized Water | 76.8 |
| Cellulose Gum | 0.4 |
| Cetyl Hydroxyethylcellulose | 0.3 |
| Raspberry Ketone | 0.5 |
| Phenylpropanol | 0.8 |
| Bis-Stearoxydimethylsilane (and) Stearyl Alcohol (and) | 2 |
| Dimethicone | |
| PEG-100 Stearate (and) Glyceryl Stearate | 1 |
| Butyrospermum Parkii (Shea) Butter | 2 |
| Cetyl Lactate | 4 |
| Isocetyl Stearoyl Stearate | 3 |
| Octyldodecyl Stearate | 3 |
| Ethylhexyl Palmitate | 4 |
| CBD | 1 |
| CBG | 1 |
| PTDTBH | 0.2 |

### Example 7: Body Cream

| Ingredient | Level(%) |
|---|---|
| De-ionized Water | 73.3 |
| Cellulose gum | 0.8 |
| Dehydroxanthan Gum | 0.2 |
| Glycerin | 1 |
| Propanediol | 1 |
| Glyceryl Stearate | 4 |
| Glyceryl Stearate Citrate | 4 |
| Cetearyl Alcohol | 4 |
| Dicaprylyl Carbonate | 3 |
| Diisostearoyl Polyglyceryl-3 Dimer Dilinoleate (and) | 2 |
| Caprylic/Capric Triglyceride | |
| Butyrospermum Parkii (Shea Butter) | 2 |
| Olea Europaea (Olive) Fruit Oil | 2 |
| CBD | 1 |
| CBG | 1 |
| PTDTBH | 0.2 |

### Example 8: Body Cream

| Ingredient | Level(%) |
|---|---|
| De-ionized Water | 81.5 |
| Glycerin | 2 |
| Ceteryl Alcohol (and) Dicetyl Phosphate (and) Ceteth-10 | 5 |
| Phosphate | |
| (Cetyl Alcohol (and) Isostearyl Isostearate (and) Potassium | 5 |
| Cetyl Phosphate (and) Cetyl Behenate (and) Behenic Acid | |
| PPG 2 Myristyl Ether Propionate | 3 |
| (Sodium Polycrylate (and) Ethylhexyl Cocoate (and) PPG-3 | 0.8 |
| Benzyl Ether Myristate (and) Polysobate 20 | |
| DMDM Hydantoin | 0.5 |
| CBD | 1 |
| CBG | 1 |
| PTDTBH | 0.2 |

### Example 9: Body Butter

| Ingredient | Level(%) |
|---|---|
| De-ionized Water | 65.6 |
| Disodium EDTA | 0.1 |
| Acrylates/C10-30 Alkyl Acrylate Crosspolymer | 0.1 |
| Carbomer | 0.25 |
| Glycerin | 2 |
| Butylene Glycol | 4 |
| Cetyl Ethylhexanoate | 7 |
| Helianthus Annuus (Sunflower) Seed Oil | 2 |
| Dimethicone (50 cSt) | 2 |
| Stearic Acid | 12 |
| PEG-20 Methyl Glucose Sesquistearate | 0.5 |
| Butyrospermum Parkii | 1 |
| Triethanolamine (99%) | 1.25 |
| CBD | 1 |
| CBG | 1 |
| PTDTBH | 0.2 |

### Example 10: Revitalizing Location

| Ingredient | Level(%) |
|---|---|
| De-ionized Water | 65.1 |
| Glycerin | 10 |
| Potassium Cetyl Phosphate | 0.6 |
| Phenylbenzimidazole Sulfonic Acid | 1 |
| Sunflower Seed Oil Sorbitol Esters | 2 |
| Caprylic/Capric Triglyceride | 4 |
| Dicaprylyl Ether | 4 |
| Behenyl Alcohol | 4 |
| Stearyl Alcohol | 4 |
| Butyl Methoxydibenzoylmethane | 1 |
| Biosaccharide Gum-1 | 1 |
| Ubiquinone | 0.1 |
| Triethanol Phenoxyethanol and Methylparaben and | 1 |
| Ethylparaben and Butylparaben and Propylenparaben | |
| and Isobutylparaben | |
| CBD | 1 |
| CBG | 1 |
| PTDTBH | 0.2 |

### Example 11: Hand Location

| Ingredient | Level(%) |
|---|---|
| De-ionized Water | 72 |
| Glycerin | 5 |
| Water (and) Montmorillonite | 4 |
| Xanthan Gum | 0.3 |
| Disodium Sulfosuccinate Decylglucoside Crosspolymer | 6 |
| Sorbitan Oleate Decylglucoside Crosspolymer | 0.5 |
| Lauryl Glucosides Betaine Crosspolymer | |
| Cetearyl Alcohol | 1 |
| Squalane | 5 |
| Propyl Citrate | 2 |
| CBD | 1 |
| CBG | 1 |
| PTDTBH | 0.2 |

### Example 12: Hand Location

| Ingredient | Level(%) |
|---|---|
| De-ionized Water | 79.8 |
| Bentonite Clay | 2.5 |
| Xanthan Gum | 0.5 |
| Cetearyl Wheat Straw Glycosides (and) Cetearyl Alcohol | 5 |
| Simmondsia Chinensis (Jojoba) Seed Oil | 5 |
| Butyrospermum Parkii (Shea) Butter | 2 |
| Theobroma Cacao (Cocoa) Seed Butter | 2 |
| Dehydroacetic Acid (and) Benzyl alcohol | 1 |
| CBD | 1 |
| CBG | 1 |
| PTDTBH | 0.2 |

### Example 13: Body Location

| Ingredient | Level(%) |
|---|---|
| De-ionized Water | 65.8 |
| Glycerin | 8 |
| Panthenol | 3 |
| Benzyl Alcohol (and) Dehydroacetic Acid | 8 |
| Cetearyl Olivate, Sorbitan Olivate | 1 |
| Cetyl Alcohol | 2 |
| Coco-Caprylate | 5 |
| Caprylic/Capric Triglyceride | 3 |
| Butyrospermum Parkii (Shea) Butter | 2 |
| CBD | 1 |
| CBG | 1 |
| PTDTBH | 0.2 |

### Example 14: Moisturizing Hand Lotion

| Ingredient | Level(%) |
|---|---|
| Water | 66.1 |
| Hydroxyethyl Urea | 14 |
| Triethyl citrate | 0.3 |
| Panthenol | 0.6 |
| Glyceryl Stearate SE | 6 |
| Cetearyl Alcohol | 2 |
| Fatty acids, C16-18 | 2 |
| Ethylhexyl Stearate | 3 |
| Mineral Oil | 3 |
| Phenoxyethanol (and) Ethylhexylglycerin | 0.8 |
| CBD | 1 |
| CBG | 1 |
| PTDTBH | 0.2 |

### Example 15: Body Cream

| Ingredient | Level(%) |
|---|---|
| Water | 82.8 |
| Glycerin | 3 |
| Polyglyceryl-2-Sesquiisostearate | 0.5 |
| Trilaureth-4 Phosphate | 2 |
| Paraffinum Liquidum | 3 |
| Hydrogenated Ethylhexyl Olivate (and) Hydrogenated | 2 |
| Olive Oil Unsaponifiables | |
| Trideceth-9 PG-Amodimethicone (and) Trideceth-12 | 0.5 |
| Cetearyl Alcohol | 2 |
| Ammonium Acryloyldimethyltaurate/VP Copolymer | 1 |
| Sorbitan Caprylate (and) Benzoic Acid (and)1,3- | 1 |
| PropaneDiol | |
| CBD | 1 |
| CBG | 1 |
| PTDTBH | 0.2 |

### Example 16: Body Cream

| Ingredient | Level(%) |
|---|---|
| Water | 66.8 |
| Xanthan Gum | 1 |
| Potassium Cocoate | 2 |
| Cocos Nucifera (Coconut) Oil | 20 |
| Caprylic/Capric Triglyceride | 5 |
| Simmondsia chinensis (Jojoba) Seed Oil | 2 |
| Phenoxyethanol (and) (and) Ethylhexylglycerin | 1 |
| CBD | 1 |
| CBG | 1 |
| PTDTBH | 0.2 |

### Example 17: Body Cream

| Ingredient | Level(%) |
|---|---|
| Water | 74.65 |
| Carbomer | 0.15 |
| Dicaprate, Tridecyl Trimellitate, Tridecyl Stearate | 10 |
| Isopropyl Palmitate | 6 |
| Stearic Acid | 2 |
| Cetyl Alcohol | 1 |
| Glyceryl Stearate, PEG-100 Stearate | 1 |
| Phenyl Trimethicone, Polyphenylmethylsiloxane | 1 |
| Cyclopentasiloxane | |
| Cyclopentasiloxane | 1 |
| Phenoxyethanol (and) Ethylhexylglycerin | 1 |
| CBD | 1 |
| CBG | 1 |
| PTDTBH | 0.2 |

### Example 18: Body Butter

| Ingredient | Level(%) |
|---|---|
| Water | 78 |
| Glycerine | 2 |
| Xanthan Gum | 0.3 |
| Behenyl Alcohol (and) Glyceryl Stearate (and) Glyceryl | 3 |
| Stearate Citrate (and) Disodium Ethylene Dicocamide | |
| PEG-15 Disulfate | |
| Cetearyl Alcohol | 2 |
| C12-13 Alkyl Lactate | 3 |
| Dicaprylyl Ether | 3 |
| Carbomer | 5 |
| Sodium Hydroxide | 0.5 |
| Phenoxyethanol (and) Ethylhexylglycerin | 1 |
| CBD | 1 |
| CBG | 1 |
| PTDTBH | 0.2 |

### Example 19: Facial Cream

| Ingredient | Level(%) |
|---|---|
| Water | 82.8 |
| Disodium EDTA | 0.1 |
| Tremella Fuciformis Polysaccharide (and) 1,2-Hexanediol | 5 |
| Xanthan Gum | 0.4 |
| Ammonium Polyacryloyldimethyl Taurate | 0.5 |
| Water (and) Acer Rubrum Extract (and) Glycerin | 6 |
| Undecane (and) Tridecane (and) Hydrogenated Olive Oil | 2 |
| Unsaponifiables (and) CocoCaprylate/Caprate | |
| CBD | 1 |
| CBG | 1 |
| PTDTBH | 0.2 |

### Example 20: Facial Cream

| Ingredient | Level(%) |
|---|---|
| Water | 72.2 |
| DMDM Hydantoin | 0.6 |
| Argania Spinosa Kernel Oil (and) Tocopheryl Acetate | 5 |
| (and) Bisabolol | |
| Octyldodecanol | 5 |
| Sodium Laureth Sulfate | 2 |
| Shea Butter | 2 |
| Glycerin | 8 |
| Dimethicone (and) Water (and) Glycerin (and) Pentylene | 2 |
| Glycol (and) Dimethicone/Vinyl Dimethicone Crosspolymer | |
| (and) Amodimethicone (and) Carbomer (and) Phenoxyethanol | |
| (and) Sodium Hydroxide (and) Disodium EDTA | |
| Phenoxyethanol (and) Ethylhexylglycerin | 1 |
| CBD | 1 |
| CBG | 1 |
| PTDTBH | 0.2 |

### Example 21: Facial Cream

| Ingredient | Level(%) |
|---|---|
| Water | 80.7 |
| Disodium EDTA | 0.1 |
| Glycerin | 3 |
| Dimethicone | 1 |
| PEG-20 Methyl Glucose Sesquistearate | 2 |
| Methyl Glucose Sesquistearate | 1 |
| Isodecyl Neopentanoate | 1 |
| Diisopropyl Adipate | 3 |
| Cyclopentasiloxane | 5 |
| Phenoxyethanol (and) Benzoic Acid (and) Dehydroacetic Acid | 1 |
| CBD | 1 |
| CBG | 1 |
| PTDTBH | 0.2 |

### Example 22: Facial Cream

| Ingredient | Level(%) |
|---|---|
| Water | 68.2 |
| Disodium EDTA | 0.1 |
| Glycerin | 3 |
| Glyceryl Caprylate | 1 |
| Triolein | 10 |
| Isostearyl Isostearate | 10 |
| Glyceryl Stearate | 3 |
| Cetearyl Alcohol | 1.5 |
| Phenoxyethanol (and) Ethylhexylglycerin | 1 |
| CBD | 1 |
| CBG | 1 |
| PTDTBH | 0.2 |

### Example 23: Balance Lotion

| Ingredient | Level(%) |
|---|---|
| Water | 67.1 |
| Xanthan Gum | 0.7 |
| Glycerin | 3 |
| Squalane (and) Glycerin (and) Sodium Surfactin | 12 |
| Cannabis Sativa (Hemp) Seed Oil | 12 |
| Behenyl Alcohol (and) Polyglyceryl-3 Stearate | 2 |
| Phenoxyethanol (and) Ethylhexylglycerin | 1 |
| CBD | 1 |
| CBG | 1 |
| PTDTBH | 0.2 |

### Example 24: Care Lotion

| Ingredient | Level(%) |
|---|---|
| Water | 82.6 |
| Acrylates/C10-30 Alkyl Acrylate Crosspolymer | 0.7 |
| Glycerin | 3 |
| PEG-15 Glyceryl Isosterate | 1.5 |
| Sodium Cocoyl Glutamate | 8 |
| Cichorium intubybus (chicory) leaf extract, maltodextrin | 1 |
| Phenoxyethanol (and) Ethylhexylglycerin | 1 |
| CBD | 1 |
| CBG | 1 |
| PTDTBH | 0.2 |

### Example 25: Cleansing Balm

| Ingredient | Level(%) |
|---|---|
| Olea Europaea (Olive) Fruit Oil/Sunflower Oil/ Sweet | 48.6 |
| Almond Oil | |
| Squalane | 6 |
| Hemp Seed Oil | 1 |
| Caprylic/Capric Triglyceride/Coconut Oil | 4 |
| Beeswax/Candelilla Wax | 6 |
| Cetearyl Alcohol | 2.5 |
| Glyceryl Stearate/Glycol Stearate/Glycol Distearate | 6 |
| Lecithin/Lanolin | 1 |
| PEG-7 Glyceryl Cocoate | 20 |
| Tocopheryl Acetate | 2 |
| Phenoxyethanol and Ethylhexyl Glycerin/Caprylhydroxamic | 0.2 |
| Acid/Caprylyl Glycol | |
| Fragrance | 0.5 |
| CBD | 1 |
| CBG | 1 |
| PTDTBH | 0.2 |

### Example 26: Lotion

| Ingredient | Level(%) |
|---|---|
| De-ionized Water | 73.03 |
| Zinc PCA | 0.75 |
| Propylene Glycol | 0.39 |
| Glycerin | 2 |
| Xanthan Gum | 0.1 |
| Squalane | 6 |
| C12-15 Alkyl Benzoate | 4 |
| Coco-Caprylate | 2 |
| Cetearyl Olivate + Sorbitan Olivate | 0.5 |
| Cetyl Alcohol | 0.25 |
| Tocopheryl Acetate | 0.5 |
| Polyacrylate Crosspolymer-6 | 1 |
| Propanediol | 5 |
| Phenoxyethanol | 0.9 |
| Ethylhexylglycerin | 0.1 |
| Bakuchiol | 0.5 |
| Propylene Glycol + Ethyl Menthane Carboxamide + | 0.5 |
| Menthyl Lactate + Methyl Diisopropyl Propionamide + | |
| Hydroxypropylcellulose + Isopropyl Palmitate + | |
| Phenoxyethanol + Silica + Ethylhexylglycerin | |
| Camellia Sinensis Leaf Extract + Maltodextrin - Organic | 0.05 |
| La Mer Type + Bergamot Fragrance | 0.1 |
| CI 42090 Ext FD&C Blue 1, 0.1% | 0.05 |
| CI 19140 FD&C Yellow 5, 1% | 0.08 |
| CBD | 1 |
| CBG | 1 |
| PTDTBH | 0.2 |

### Example 27: Facial Lotion

| Ingredient | Level(%) |
|---|---|
| De-ionized Water | 81.13 |
| Glycerin | 2.93 |
| Polyacrylate Crosspolymer-6 | 0.29 |
| Sodium Polyacrylate | 0.24 |
| Caprylic/Capric Triglyceride | 3.91 |
| Squalane | 1.96 |
| Cetearyl Olivate (and) Sorbitan Olivate | 2.45 |
| Cetyl Alcohol | 0.98 |
| Dicaprylyl Carbonate | 2.93 |
| Phenoxyethanol and Ethylhexyl Glycerin | 0.98 |
| CBD | 1 |
| CBG | 1 |
| PTDTBH | 0.2 |

### Example 28: Facial Lotion

| Ingredient | Level(%) |
|---|---|
| De-ionized Water | 81.21 |
| Glycerin | 2.94 |
| Polyacrylate Crosspolymer-6 | 0.29 |
| Sodium Polyacrylate | 0.24 |
| Caprylic/Capric Triglyceride | 3.92 |
| Squalane | 1.96 |
| Cetearyl Olivate (and) Sorbitan Olivate | 2.45 |
| Cetyl Alcohol | 0.98 |
| Dicaprylyl Carbonate | 2.94 |
| Phenoxyethanol and Ethylhexyl Glycerin | 0.98 |
| CBD | 1 |
| CBG | 1 |
| PTDTBH | 0.1 |

### Example 29: Facial Lotion

| Ingredient | Level(%) |
|---|---|
| De-ionized Water | 81.25 |
| Glycerin | 2.94 |
| Polyacrylate Crosspolymer-6 | 0.29 |
| Sodium Polyacrylate | 0.24 |
| Caprylic/Capric Triglyceride | 3.92 |
| Squalane | 1.96 |
| Cetearyl Olivate (and) Sorbitan Olivate | 2.45 |
| Cetyl Alcohol | 0.98 |
| Dicaprylyl Carbonate | 2.94 |
| Phenoxyethanol and Ethylhexyl Glycerin | 0.98 |
| CBD | 1 |
| CBG | 1 |
| PTDTBH | 0.05 |

### Example 30: Facial Lotion

| Ingredient | Level(%) |
|---|---|
| De-ionized Water | 81.28 |
| Glycerin | 2.94 |
| Polyacrylate Crosspolymer-6 | 0.29 |
| Sodium Polyacrylate | 0.24 |
| Caprylic/Capric Triglyceride | 3.92 |
| Squalane | 1.96 |
| Cetearyl Olivate (and) Sorbitan Olivate | 2.45 |
| Cetyl Alcohol | 0.98 |
| Dicaprylyl Carbonate | 2.94 |
| Phenoxyethanol and Ethylhexyl Glycerin | 0.98 |
| CBD | 1 |
| CBG | 1 |
| PTDTBH | 0.01 |

### Example 31: Facial Lotion

| Ingredient | Level(%) |
|---|---|
| De-ionized Water | 80.46 |
| Glycerin | 2.91 |
| Polyacrylate Crosspolymer-6 | 0.29 |
| Sodium Polyacrylate | 0.24 |
| Caprylic/Capric Triglyceride | 3.88 |
| Squalane | 1.94 |
| Cetearyl Olivate (and) Sorbitan Olivate | 2.43 |
| Cetyl Alcohol | 0.97 |
| Dicaprylyl Carbonate | 2.91 |
| Phenoxyethanol and Ethylhexyl Glycerin | 0.97 |
| CBD | 1 |
| CBG | 1 |
| BHT | 1 |

### Example 32: Facial Lotion

| Ingredient | Level(%) |
|---|---|
| De-ionized Water | 81.25 |
| Glycerin | 2.94 |
| Polyacrylate Crosspolymer-6 | 0.29 |
| Sodium Polyacrylate | 0.24 |
| Caprylic/Capric Triglyceride | 3.92 |
| Squalane | 1.96 |
| Cetearyl Olivate (and) Sorbitan Olivate | 2.45 |
| Cetyl Alcohol | 0.98 |
| Dicaprylyl Carbonate | 2.94 |
| Phenoxyethanol and Ethylhexyl Glycerin | 0.98 |
| CBD | 1 |
| CBG | 1 |
| Propyl Gallate | 0.05 |

### Example 33: Facial Lotion

| Ingredient | Level(%) |
|---|---|
| De-ionized Water | 81.13 |
| Glycerin | 2.93 |
| Polyacrylate Crosspolymer-6 | 0.29 |
| Sodium Polyacrylate | 0.24 |
| Caprylic/Capric Triglyceride | 3.91 |
| Squalane | 1.96 |
| Cetearyl Olivate (and) Sorbitan Olivate | 2.45 |
| Cetyl Alcohol | 0.98 |
| Dicaprylyl Carbonate | 2.93 |
| Phenoxyethanol and Ethylhexyl Glycerin | 0.98 |
| CBD | 1 |
| CBG | 1 |
| Propyl Gallate | 0.2 |

### Example 34: Facial Lotion

| Ingredient | Level(%) |
|---|---|
| De-ionized Water | 79.63 |
| Glycerin | 2.88 |
| Polyacrylate Crosspolymer-6 | 0.29 |
| Sodium Polyacrylate | 0.24 |
| Caprylic/Capric Triglyceride | 3.84 |
| Squalane | 1.92 |
| Cetearyl Olivate (and) Sorbitan Olivate | 2.40 |
| Cetyl Alcohol | 0.96 |
| Dicaprylyl Carbonate | 2.88 |
| Phenoxyethanol and Ethylhexyl Glycerin | 0.96 |
| CBD | 1 |
| CBG | 1 |
| Vitamine A | 2 |

### Example 35: Facial Lotion

| Ingredient | Level(%) |
|---|---|
| De-ionized Water | 79.63 |
| Glycerin | 2.88 |
| Polyacrylate Crosspolymer-6 | 0.29 |
| Sodium Polyacrylate | 0.24 |
| Caprylic/Capric Triglyceride | 3.84 |
| Squalane | 1.92 |
| Cetearyl Olivate (and) Sorbitan Olivate | 2.40 |
| Cetyl Alcohol | 0.96 |
| Dicaprylyl Carbonate | 2.88 |
| Phenoxyethanol and Ethylhexyl Glycerin | 0.96 |
| CBD | 1 |
| CBG | 1 |
| Vitamine E | 2 |

### Example 36: Facial Lotion

| Ingredient | Level(%) |
|---|---|
| De-ionized Water | 77.14 |
| Glycerin | 2.79 |
| Polyacrylate Crosspolymer-6 | 0.28 |
| Sodium Polyacrylate | 0.23 |
| Caprylic/Capric Triglyceride | 3.72 |
| Squalane | 1.86 |
| Cetearyl Olivate (and) Sorbitan Olivate | 2.33 |
| Cetyl Alcohol | 0.93 |
| Dicaprylyl Carbonate | 2.79 |
| Phenoxyethanol and Ethylhexyl Glycerin | 0.93 |
| CBD | 1 |
| CBG | 1 |
| Grape Seed Oil | 2.5 |
| Olive Oil | 2.5 |

According to some embodiments of the present disclosure, both CBD and CBG are included in the cosmetic composition (i.e., the third composition), and the presence of both CBD and CBG may improve the comprehensive effectiveness of the cosmetic composition. In some embodiments, a weight ratio of CBD and CBG is 1:1; such a ratio would be suitable for some formula, making the cosmetic composition more balanced in terms of anti-inflammatory and/or antioxidant effects than a cosmetic composition including only CBD, only CBG, or both CBD and CBG with other weight ratios. For example, in some embodiments, each of the CBD and CBG, at an ideal level, may be 1% by weight of the cosmetic composition. In addition, by adding the stabilizing agent in the cosmetic composition, the cannabinoids (e.g., CBD and CBG) in the cosmetic composition can be protected from being oxidized or the oxidization rate of the cannabinoids (e.g., CBD and CBG) in the cosmetic composition can be significantly reduced, thereby making the cosmetic composition more stable. PTDTBH would be one of the optimal stabilizing agents for stabilizing the cannabinoids in the cosmetic composition. PTDTBH at a level greater than 0.05% (e.g., at 0.1%, 0.2%), especially at 0.2%, by weight in the cosmetic composition may improve the stability of the cannabinoids in the cosmetic composition to a desired extent. In some embodiments, besides the CBD, CBG, and the stabilizing agent, the cosmetic composition would be added with any other different active agents as well as the active agents do not affect or affect as little as possible the function of the stabilizing agent for the CBD and/or CBG in the cosmetic composition, which further improves the comprehensive effectiveness of the cosmetic composition.

Stability Test Method

The present disclosure provides an HPLC method which is a suitable manner for determining the loss of cannabinoids in a composition, which reflects the stability of the cannabinoids in the composition.

A sufficient quantity of a test composition (e.g., the first composition or the second composition) is placed in a controlled environment chamber/room set at a preset temperature (e.g., 45° C.) for a preset time (e.g., 1 month). After the preset time, the composition is removed from the controlled environment.

The stability of cannabinoids in the test composition is determined on a % w/w basis by HPLC (isocratic elution) as follows. The HPLC column(s) is conditioned in accordance with conventional practices.

Chromatographic Conditions

1. Column: C18 (5 microns), 150 mm×4.6 mm

2. Mobile Phase (Eluent): a mixture of methanol, acetonitrile, and water (52:30:18 v/v)

3. Column temperature: approximately 25° C. (Ambient)

4. UV wavelength: 228 nm

5. Injection volume: 20 microliters

6. Flow rate: 1.8 ml/min

7. Run time: approximately 6 minutes

8. Cannabinoid retention time: approximately 5 minutes (e.g., 4.72 minutes)

Mobile Phase Preparation

1 L of Mobile Phase (Eluent) is prepared by mixing 520 mL of methanol, 380 mL of acetonitrile, and 180 mL of water, adjusting the mixture to a pH of 4.5 with acetic acid, and further filtering the adjusted mixture through a membrane filter (47 mm membrane, 0.45 μm pore size, nylon) and degassed in an ultrasonic bath before use.

External Standard Preparation (prepare fresh on the day of use)

A reference standard solution of cannabinoids in methanol (1 mg/mL) is prepared by dissolving 25 mg of cannabinoids the same as that contained in the test composition in 25 mL of methanol without being exposed to light. Concentrations between 1-150 μg/m L are prepared by transferring a 5 mL aliquot of the reference standard solution to a 50 mL amber flask and diluting to volume with the mobile phase.

HPLC Sample Preparation

The test composition is mixed with ethanol thoroughly before testing according to a preset concentration (100 μg/m L). The mixed composition is stored at −20° C. without being exposed to light. Before use, approximately 1 ml of the mixed composition is filtered into an auto-sampler vial using a syringe filter.

(A)/(B)×(B)/(W)×(DF)×100=cannabinoid,% w/w

where, A=peak area of cannabinoid for the sample, B=peak area of cannabinoid for the calibration, C=cannabinoid standard weight in mg, W=sample weight, mg, DF=Dilution Factor (e.g., 0.1).

The present disclosure also provides stability tests for CBD/CBG, which can reflect the stability of the cannabinoids in a cosmetic composition (e.g., the third composition).

Seven samples were prepared for the stability tests. The seven samples to be tested were composed of the same base formula with different levels, CBD and/or CBG with the same level, and antioxidants with different types and/or levels. A positive control sample was also prepared composed of the same base formula and CBD and CBD without a stabilizing agent and the CBD and CBG were at the same levels as that in the seven samples. The seven samples (i.e., S1-S7) and the positive control sample (i.e., S8) are listed as follows, illustrating the levels of ingredient s by weight in the samples.

| | S1 | S2 | S3 | S4 | S5 | S6 | S7 | S8 |
|---|---|---|---|---|---|---|---|---|
| Base | 96% | 96% | 97% | 93% | 97.95% | 97.9% | 97.8 | 98% |
| Formula | | | | | | | | |
| CBD | 1% | 1% | 1% | 1% | 1% | 1% | 1% | 1% |
| CBG | 1% | 1% | 1% | 1% | 1% | 1% | 1% | 1% |
| Vitamin A | 2% | — | — | — | — | — | — | — |
| Vitamin E | — | 2% | — | — | — | — | — | — |
| BHT | — | — | 1% | — | | — | — | — |
| Grape | — | — | — | 2.5% | — | — | — | — |
| Seed Oil | | | | | | | | |
| Olive Oil | — | — | — | 2.5% | — | — | — | — |
| PTDTBH | — | — | — | — | 0.05% | 0.1% | 0.2% | — |

Ingredients of the base formula are listed as follows, illustrating the levels of the ingredients by weight in the base formula.

| Ingredient | Level (%) |
|---|---|
| Water | 82.95 |
| Glycerin | 3 |
| Polyacrylate Crosspolymer-6 | 0.3 |
| Sodium Polyacrylate | 0.25 |
| Caprylic/Capric Triglyceride | 4 |
| Squalane | 2 |
| Cetearyl Olivate (and) Sorbitan Olivate | 2.5 |
| Cetyl Alcohol | 1 |
| Dicaprylyl Carbonate | 3 |
| Phenoxyethanol and Ethylhexyl Glycerin | 1 |

The seven samples S1-S7 were put in stability in glass jars for 3 months in an oven (45° C.) to obtain seven processed samples 51′-S7′. During the 3 months, the seven samples S1-S7 were checked in color every week. The sample S1 showed a color change after 1 week. Samples S2 and S4 showed slight color changes after 1 week. Samples S3, S5, S6, and S7 showed no color change (or no obvious color change) even after 9 weeks. Accordingly, CBD and CBG in the samples S3, S5, S6, and S7 are indicated to be more stable than that in the samples S1, S2, and S4.

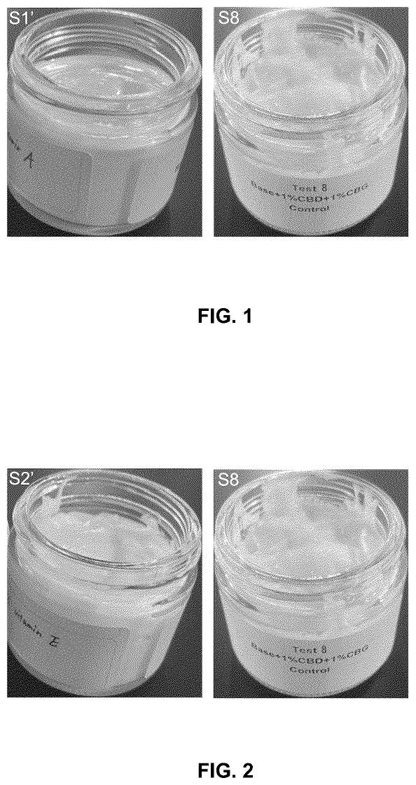

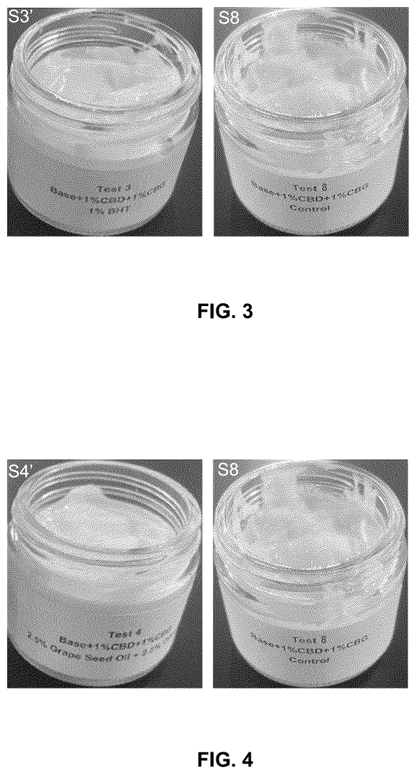

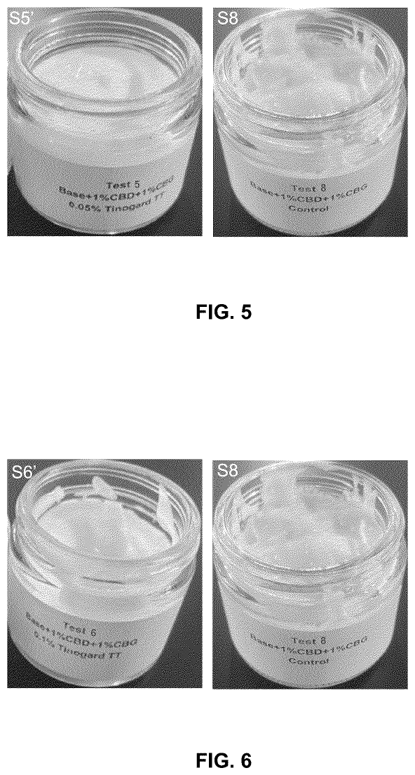

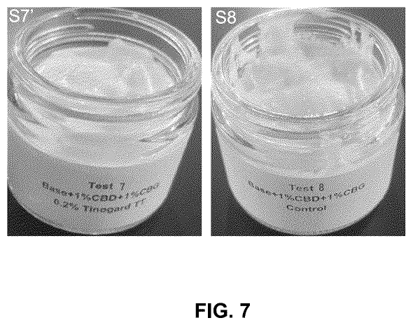

FIGS. 1-7 show images of the processed samples 51′-S7′ with an image of the positive control sample S8 respectively. As shown, the positive control sample S8 was white, which indicates that the positive control sample is not or substantially not oxidized. The original colors of the samples S1-S7 were close to the color of the positive control sample. Each of the processed samples 51′-S7′ was compared with the positive control sample S8 in color. If a processed sample of the processed samples S1′-S7′ is darker than the positive control sample, the processed sample may be determined to be oxidated after 3 months. The more the color difference between the processed sample and the positive control sample S8 is, the less stable CBD and CBG in the processed sample may be. As shown in FIGS. 1-7, the color differences between the processed sample S1′-S7′ and the positive control sample S8 can be ranked in descending order as: the color difference between S1′ and S8>the color difference between S4′ and S8>the color difference between S2′ and S8>the color difference between S3′ and S8>the color difference between S5′ and S8>the color difference between S6′ and S8>the color difference between S7′ and S8. Accordingly, CBD and CBG in the samples with PTDTBH are more stable, and the more PTDTBH a sample contains, the more stable CBD and CBG in the sample may be.

In addition, the processed sample S4′ and the processed sample S7′ were measured using a GC-MS device (GC-7890A and MSD-5975C produced by Agilent Technologies) according to the analyzed test provided in “Aviv, Amirav, et al. Cannabis and its cannabinoids analysis by gas chromatography-mass spectrometry with Cold EI. March 2021 Journal of Mass Spectrometry 56(6)” to determine levels of CBD and CBG in the processed samples S4′ and S7′. The measurement results are shown as follows.

The measurement result of the processed sample S4′

| Analyte | LOQ (limit of quantitation) | Mass (%) |
|---|---|---|
| THCa | 0.08 | ND |
| Δ9-THC | 0.04 | ND |
| Δ8-THC | 0.04 | ND |
| THCV | 0.04 | ND |
| CBDa | 0.08 | ND |
| CBD | 0.04 | 0.80 |
| CBDV | 0.04 | ND |
| CBN | 0.04 | ND |
| CBGa | 0.08 | ND |
| CBG | 0.04 | 0.79 |
| CBC | 0.04 | ND |
| Total | — | 1.60 |

It can be seen from the measurement result of the processed sample S4′, under the action of grape oil and olive oil, CBD and CBG remained in the processed sample S4′ after three months of storage at 45° C. and in light is equal to 80% by weight of the original CBD and CBG in the sample S4.

The measurement result of the processed sample S7′

| Analyte | LOQ (limit of quantitation) | Mass (%) |
|---|---|---|
| THCa | 0.08 | ND |
| Δ9-THC | 0.04 | ND |
| Δ8-THC | 0.04 | ND |
| THCV | 0.04 | ND |
| CBDa | 0.08 | ND |
| CBD | 0.04 | 0.91 |
| CBDV | 0.04 | ND |
| CBN | 0.04 | ND |
| CBGa | 0.08 | ND |
| CBG | 0.04 | 0.85 |
| CBC | 0.04 | ND |
| Total | — | 1.75 |

It can be seen from the measurement result of the processed sample S7′, under the action of PTDTBH, CBD and CBG remained in the processed sample S7′ after three months of storage at 45° C. and in light is equal to 87.5% by weight of the original CBD and CBG in the sample S7.

Further, sample S9 was also prepared with only the same base formula as samples S1-S8 with 99% by weight of the sample S9 and CBD with 1% by weight of the sample S9. The sample S9 was white. The sample S9 was measured using the GC-MS device similar to the measurement of the processed samples S4′ and S7′. The measurement result of the sample S9 is shown as follows.

| Analyte | LOQ (limit of quantitation) | Mass (%) |
|---|---|---|
| THCa | 0.08 | ND |
| Δ9-THC | 0.04 | ND |
| Δ8-THC | 0.04 | ND |
| THCV | 0.04 | ND |
| CBDa | 0.08 | ND |
| CBD | 0.04 | 0.89 |
| CBDV | 0.04 | ND |
| CBN | 0.04 | ND |
| CBGa | 0.08 | ND |
| CBG | 0.04 | 0.85 |
| CBC | 0.04 | ND |
| Total | — | 0.89 |

It can be seen from the measurement result of the sample S9, CBD and CBG remained in the white sample S9 is equal to 89% by weight of the original CBD and CBG in the sample S9.

Accordingly, CBD and CBG in the sample S9 are more than CBD and CBG in the processed sample S7′, and CBD and CBG in the processed sample S7′ are more than CBD and CBG in processed S4′, which are consistent with the color checked results in FIGS. 1-7.

It should be noted that the above descriptions are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations, therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software-only solution, e.g., an installation on an existing server or mobile device.

Similarly, it should be appreciated that in the foregoing description of embodiments of the present disclosure, various features are sometimes grouped together in a single embodiment, figure, or description thereof to streamline the disclosure aiding in the understanding of one or more of the various embodiments. This method of disclosure, however, is not to be interpreted as reflecting an intention that the claimed subject matter requires more features than are expressly recited in each claim. Rather, claim subject matter lies in less than all features of a single foregoing disclosed embodiment.

What is claimed is:

1. A stable cosmetic composition, comprising:
one or more cannabinoids at 0.05%-10% in total by weight of the composition;
an emulsifier at 0.02%-20% by weight of the composition; and
a stabilizing agent which includes an antioxidant.

2. The composition of claim 1, wherein the one or more cannabinoids include cannabidiol (CBD), cannabigerol (CBG), or a derivative thereof.

3. The composition of claim 1, wherein the one or more cannabinoids are at 0.5%-5% in total by weight of the composition.

4. The composition of claim 1, wherein the one or more cannabinoids are at 1%-3% in total by weight of the composition.

5. The composition of claim 1, wherein the one or more cannabinoids include CBD at about 1% by weight of the composition and CBG at about 1% by weight of the composition.

6. The composition of claim 1, wherein the antioxidant is pentaerythrityl tetra-di-t-butyl hydroxyhydrocinnamate (PTDTBH), butylated hydroxytoluene (BHT), or propyl gallate.

7. The composition of claim 1, wherein the antioxidant is at 0.01%-2% by weight of the composition.

8. The composition of claim 1, wherein the antioxidant is at 0.05%-1% by weight of the composition.

9. The composition of claim 1, wherein the antioxidant is PTDTBH at about 0.1% by weight of the composition.

10. The composition of claim 1, wherein the emulsifier is at 1%-5% by weight of the composition.

11. The composition of claim 1, wherein the emulsifier includes at least one of cetyl alcohol, cetearyl alcohol, polypropylene glycol (PPG) polyether, or polyglycerol fatty acid ester.

12. The composition of claim 1, further comprising:
a vitamin,
a liquid lipid,
a solid lipid,
a moisturizing agent,
a thickener,
a fragrance,
a coloring agent, or
an absorbent.

13. The composition of claim 12, wherein:
the vitamin is selected from the group consisting of vitamin A and vitamin E;
the liquid lipid is selected from the group consisting of triglyceride, isosorbide derivatives, and long chain straight alkane;
the solid lipid is selected from the group consisting of fatty alcohol ester, vegetable wax, and beeswax;
the moisturizing agent is selected from the group consisting of glycerin and squalane;
the thickener is selected from the group consisting of polyacrylate crosspolymer-6, proteins, alcohols, and silicones;
the fragrance is selected from the group consisting of perfume, cologne, and aftershave;
the coloring agent is selected from the group consisting of fluorescein, quinoline, triphenylmethane, titanium dioxide, iron oxide, chromium oxide, organic colorants, and particles of silver and gold; and
the absorbent is selected from the group consisting of sodium polyacrylate, kaolin, talc powder, and magnesium oxide.

14. The composition of claim 1, further comprising:
a vitamin,
a liquid lipid,
a solid lipid,
a moisturizing agent,
a thickener,
a fragrance,
a coloring agent, and
an absorbent.

15. A stable cosmetic composition, comprising:
CBD at 0.5%-1.5% by weight of the composition;
CBG at 0.5%-1.5% by weight of the composition;
an emulsifier at 0.1%-5% by weight of the composition; and
a stabilizing agent comprising PTDTBH at 0.05%-0.5% by weight of the composition, or BHT at 0.5%-2% by weight of the composition.

16. The composition of claim 15, wherein the CBD is at about 1% by weight of the composition, and the CBG is at about 1% by weight of the composition.

17. The composition of claim 15, wherein the stabilizing agent includes PTDTBH at about 0.1% by weight of the composition.

18. The composition of claim 15, wherein the emulsifier includes cetyl alcohol at 0.5%-1.5% by weight of the composition, and the composition further comprises:
water at about 75%-85% by weight of the composition;
glycerin at about 2.5%-3.5% by weight of the composition;
dicaprylyl carbonate at about 2.5%-3.5% by weight of the composition.

19. A stable cosmetic composition, comprising:
water at about 80% by weight of the composition;
CBD at about 1% by weight of the composition;
CBG at about 1% by weight of the composition;
PTDTBH at about 0.1% by weight of the composition;
glycerin at about 3% by weight of the composition;
cetyl alcohol at about 1% by weight of the composition; and
dicaprylyl carbonate at about 3% by weight of the composition.

20. The composition of claim 19, further comprising:
a vitamin,
a liquid lipid,
a solid lipid,
a moisturizing agent,
a thickener,
a fragrance,
a coloring agent, or
an absorbent.
